(19) United States
(12) Patent Application Publication
(10) Pub. No.: US 2024/0164645 A1
(43) Pub. Date: May 23, 2024

(54) APPARATUS FOR NONINVASIVE VETERINARY SCREENING AND DIAGNOSIS

(71) Applicant: INFRARED CAMERAS, INC., Beaumont, TX (US)

(72) Inventor: Gary Strahan, Beaumont, TX (US)

(21) Appl. No.: 18/508,518

(22) Filed: Nov. 14, 2023

Related U.S. Application Data
(60) Provisional application No. 63/426,856, filed on Nov. 21, 2022.

(51) Int. Cl. A61B 5/00; A61B 5/01; G06T 7/00

(52) U.S. Cl. CPC A61B 5/0064; A61B 5/015; A61B 5/743; A61B 5/7435; G06T 7/0014; A61B 2560/0431; A61B 2560/0487; G06T 2207/30004

(57) ABSTRACT

An apparatus capable of capturing an image and displaying the image. The apparatus can comprise a camera, a body, a processor, a non-transitory data storage medium, a database of animal outlines stored on the non-transitory data storage medium, and a display, wherein the display provides an animal outline to align the camera with an animal. The apparatus can display a thermographic signature of the animal and provide a historical reference thermographic signature of a specific animal having its image captured for comparison, while highlighting changes in the thermographic signatures.

### CROSS REFERENCE TO RELATED APPLICATION

The present application claims the benefit of co-pending U.S. Provisional Patent Application Ser. No. 63/426,856 filed on Nov. 21, 2022, titled “APPARATUS FOR NONINVASIVE VETERINARY SCREENING AND DIAGNOSIS”. This reference is incorporated herein in its entirety.

### FIELD

The present disclosure generally relates to a portable apparatus to aid in noninvasive veterinary screening and diagnostics.

### BACKGROUND

Veterinary screening presents its own unique impediments to veterinary practitioners. As animals cannot talk or provide context and history with regard to their medical status, veterinarians must rely on observation or invasive testing, such as blood draws or biopsies.

Much of an animal's history is discovered through the owner, including information such as the animal's diet, exercise routine, behavior, habits, elimination patterns (i.e., bowel movements and urination), lifestyle, and general health. The veterinarian can then perform a physical examination (if possible) to listen to the heartbeat, palpate areas of the body and/or lymph nodes, and inspect whether there is any evidence of discomfort for the animal.

To further complicate a veterinary exam, many animals as a natural survival instinct will hide any signs of disease or weakness, often making health issues difficult to detect in early stages.

A need exists, therefore for more detailed noninvasive screening and diagnosis of animals.

The present disclosure provides an apparatus to aid in screening and diagnosing animals for medical issues.

### BRIEF DESCRIPTION OF THE DRAWINGS

The detailed description will be better understood in conjunction with the accompanying drawings as follows:

FIG. 1 depicts an animal outline of a dog.

FIG. 2 depicts a thermographic signature of a horse.

The embodiments of the present disclosure are detailed below with reference to the listed Figures.

### DETAILED DESCRIPTION OF THE EMBODIMENTS

Before explaining the present disclosure in detail, it is to be understood that the present disclosure is not limited to the specifics of particular embodiments as described and that it can be practiced, constructed, or carried out in various ways.

While embodiments of the disclosure have been shown and described, modifications thereof can be made by one skilled in the art without departing from the spirit and teachings of the disclosure. The embodiments described herein are exemplary only and are not intended to be limiting.

Specific structural and functional details disclosed herein are not to be interpreted as limiting, but merely as a basis of the claims and as a representative basis for teaching persons having ordinary skill in the art to variously employ the present embodiments. Many variations and modifications of embodiments disclosed herein are possible and are within the scope of the present disclosure.

Where numerical ranges or limitations are expressly stated, such express ranges or limitations should be understood to include iterative ranges or limitations of like magnitude falling within the expressly stated ranges or limitations.

The word “about”, when referring to values, means plus or minus 5% of the stated number.

The use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

When methods are disclosed or discussed, the order of the steps is not intended to be limiting, but merely exemplary unless otherwise stated.

Accordingly, the scope of protection is not limited by the description herein, but is only limited by the claims which follow, encompassing all equivalents of the subject matter of the claims. Each and every claim is hereby incorporated into the specification as an embodiment of the present disclosure. Thus, the claims are a further description and are an addition to the embodiments of the present disclosure.

The inclusion or discussion of a reference is not an admission that it is prior art to the present disclosure, especially any reference that may have a publication date after the priority date of this application. The disclosures of all patents, patent applications, and publications cited herein are hereby incorporated by reference, to the extent they provide background knowledge; or exemplary, procedural or other details supplementary to those set forth herein.

The embodiments of the present disclosure generally relate to a portable apparatus to aid in veterinary screening and diagnostics.

The term “animal” as used herein typically refers to pets and domestic animals, zoo animals, farm animals, or other animals treated by veterinary practitioners, and the like. While exemplary embodiments will be described in relation to specific animals, this is not intended to be limiting in any manner. While the intended application is for non-human animals, it is contemplated that the disclosed apparatus and methods are potentially applicable for human medical treatment in the same manner as described below, and therefore humans are also included in the broadest definition of animal.

The apparatus can be capable of capturing an image and displaying the image. The apparatus can comprise a camera, a body, a processor, a non-transitory data storage medium, a database of animal outlines stored on the non-transitory data storage medium, and a display, wherein the display provides an animal outline to align the camera with an animal.

The apparatus can capture a thermographic signature of the animal and either provide a historical reference image of a specific animal having its image captured, a region reference image of the animal, or a generic reference image of a type of animal having its image captured. In embodiments, the apparatus provides a visual indication to the user highlighting a region of the animal which requires further attention or provides a visual indication to the user highlighting changes between the historical reference image and the current image.

The camera can be any camera known to persons having ordinary skill in the art capable of capturing an image and/or an infrared image. A digital camera is preferred, such that pixel information can be digitally stored and/or manipulated by computer software. In embodiments, a captured digital image can be adapted to an infrared image by utilizing pixel information.

The camera can be housed in a body with also houses a computer processor capable of executing computer instructions. In embodiments, the camera can house communication devices (i.e., a transceiver) to communicate with a computer processor housed outside the camera in a separate body. The body can also house a non-transitory data storage medium having a database of animal outlines and storing computer instructions or be in communication with such a data storage medium. Well understood computer functions are not within the scope of this disclosure and will not be addressed in detail.

The camera and/or the apparatus body can be handheld and portable. In embodiments, the camera can weigh less than five pounds, allowing for easy handling and transport. In embodiments, the camera can be integrated with a display, or can be in electronic communication with a display in a wired or wireless manner. For example, the camera can have a small display integrated while simultaneously showing the field of view on a television or monitor nearby.

The display can provide an outline of the animal whose image is being captured. In embodiments, the processor can execute computer instructions to select a specific animal outline from the non-transitory data storage medium and project the outline on the display, allowing a user of the apparatus to then aim the field of vision of the camera.

This can help the user to align the camera optimally to capture an image consistently for an animal each time the animal is photographed. In this manner, a history of the animal can be created with substantially identical images taken at various points in time. In embodiments, such photographs of the animal can be stored in electronic medical records (EMR) software for future access and assessment by doctors and veterinary practitioners.

Exemplary animal outlines include, but are not limited to a canine, a feline, a bird, a reptile, an equine, a swine, a bovine, a fish, a marine mammal, or an exotic pet. In embodiments, specific breeds or sub-species outlines can be stored and utilized.

The database of animal images can have outlines of a front view, a rear view, a left side view, a right side view, a top view, and a bottom view for each animal to allow for comprehensive records being created. In embodiments, a series of images can be taken, and the processor can execute instructions to combine these images to create a three dimensional, walk around image for manipulation and study.

When a historical image of a specific animal exists, the display can show a reference image or outline of the specific animal, wherein the specific animal images comprise at least one of: a front view, a rear view, a left side view, a right side view, a top view, or a bottom view of the specific animal. If a historical image does not exist (i.e., this is the first image capture of the animal by this practitioner, or no accessible electronic medical records exist), then a generic image or outline of the type of animal can be displayed.

A thermographic signature can be captured of the animal. Thermographic signature, as used herein, refers either actual or relative temperatures of the animal. The thermographic signature can be captured by either using an infrared camera, or by using a digital camera and manipulating pixel data to determine differences in temperature at various locations of the animal. The camera sensitivity can be adjusted to capture skin temperatures, or deeper tissue temperatures as desired by persons having ordinary skill in the art.

The thermographic signature can make use of actual temperatures, when utilizing an infrared camera which has been calibrated for the environment it is being used in, or simply make use of relative temperatures, or temperature differences between a selected reference area and other parts of the animal.

When historical data on the animal exists, differences from previous thermographic signatures can be highlighted and displayed and/or overlayed on the present thermographic signature for the user. If a historical image does not exist, an “expected” thermographic signature can be displayed and/or overlayed on the present thermographic signature.

A method of using the disclosed apparatus can include the following steps: providing a handheld camera in communication with at least one display, allowing a user to select a category and/or subcategory of animal, providing an outline of the category of animal on the screen, wherein the outline is at least one of: a front view, a rear view, a left side view, a right side view, a top view, or a bottom view, capturing an image of the animal, and identifying an area of interest on the outline of the category of animal if an area of interest exists.

An infrared image, a visible image, or both an infrared image and a visible image can be captured. Upon capture of an image, the following steps can be performed: overlaying the captured image upon the outline of the animal, comparing a captured thermographic signature with either an expected thermographic signature, or a historical thermographic signature, and highlighting an area of interest wherein at least a portion of the captured thermographic signature does not match either the expected thermographic signature or the historical thermographic signature.

### Exemplary Embodiment and Use of the Apparatus

One potential use of the disclosed apparatus would be in a veterinary clinic. An owner brings a pet in for a visit. The clinic personnel places the animal patient in an area to be photographed.

The clinician picks up the apparatus, which can be a tablet computer. The tablet computer contains all the components necessary to implement the present disclosure. In this embodiment, the tablet computer has a camera, a data storage with computer instructions for a processor to execute, a database of animal images, thermographic signatures, and outlines.

The clinician selects the type of animal from a menu to generate an outline on the display of the tablet computer. The display can also be simultaneously projected to a monitor or television in the room, allowing the owner of the animal to view the proceedings.

For example, the clinician may select “canine” from the list of outlines. In embodiments, further selections such as breed, size, or applicable characteristics can be offered.

Upon completing selections, the outline of the appropriate animal is shown on the display. The clinician aligns the outline as best as possible with the animal and captures an image. This process can be repeated to get different angles and/or viewpoints of the animal. In embodiments, the various images can be combined into a three-dimensional, manipulatable image using techniques well known in the art.

A thermographic signature of the animal, showing either actual or relative temperatures of the animal can be displayed on the screen. The clinician can select whether a skin temperature, or a deeper tissue temperature is displayed on the screen. If this is a repeat visit by the animal, a historical thermographic signature from the same viewpoint or angle can be displayed for comparison. Any variations from the previous thermographic signature can be highlighted for the clinician to investigate.

If the animal does not have accessible historical data, an expected thermographic signature with baseline value as determined by persons having ordinary skill in the art can be displayed for comparison. Any variations from the expected thermographic signature can be highlighted for the clinician to investigate.

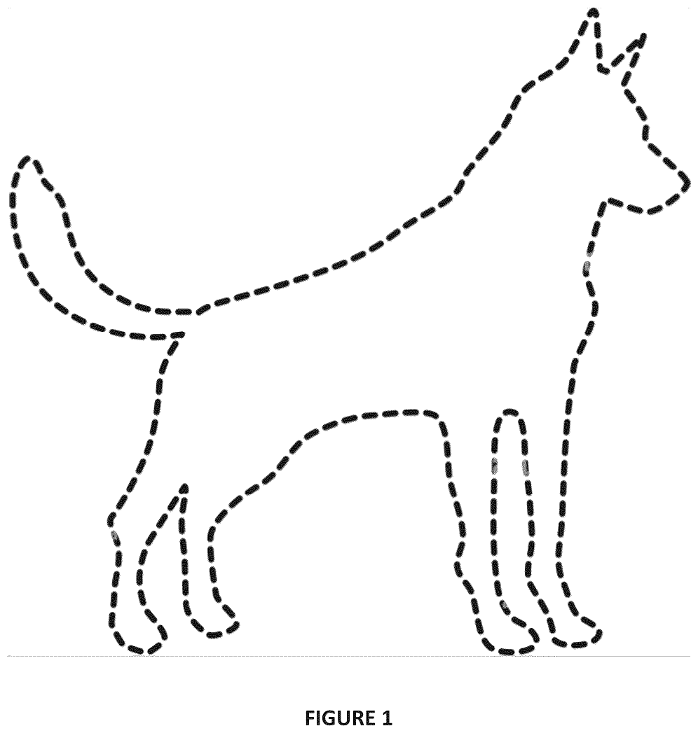

Turning now to the Figures, FIG. 1 depicts an animal outline of a dog.

The clinician can align this outline with an animal to capture an image. The outline will be superimposed on the screen when aligning a digital camera with an animal.

In embodiments, a semitransparent historical image can be within the outline. In other embodiments, a region of interest can be highlighted within the outline. In embodiments, a user can select specific historical images or regions to highlight within the outline. In embodiments, a user can also manually mark a section of the outline to be used in the future for the same animal. In other embodiments, a region or area of the outline can be automatically selected or highlighted by the apparatus based upon certain user defined criteria.

Outlines can be of a generic animal of the species, or a specific outline of the animal as created from a previous picture. By using a specific outline, weight gain or loss can be compared and/or determined.

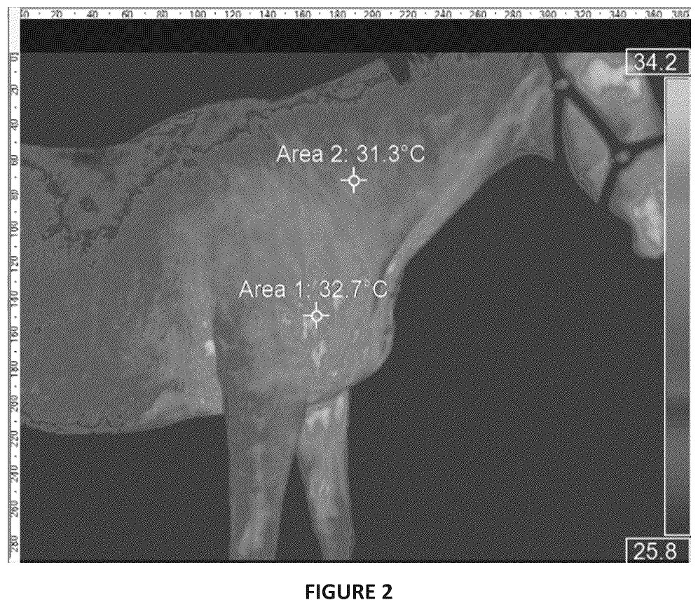

FIG. 2 depicts a thermographic signature of a horse.

Here we see a potentially infected area highlighted on the thermographic signature. This is even more evident in a color image, which would be shown to a user. Historical images containing this information can be presented alongside a live or recently captured image. In this manner, disease progression/regression or regions requiring special attention can be identified.

While the present disclosure emphasizes the presented embodiments and Figures, it should be understood that within the scope of the appended claims, the disclosure might be embodied other than as specifically enabled herein

What is claimed is:

1. An apparatus capable of capturing an image and displaying the image comprising:
a. a camera;
b. a body;
c. a processor;
d. a non-transitory data storage medium having computer instructions stored thereon for execution by the processor;
e. a database of animal outlines stored on the non-transitory data storage medium; and
f. a display, wherein the display:
(i) provides an animal outline to align the camera with an animal, wherein the animal is having its image captured, and further wherein the apparatus captures a thermographic signature of the animal;
(ii) provides a historical reference image of a specific animal having its image captured, or a region reference image of the animal, or a generic reference image of a type of animal having its image captured; and
(iii) provides a visual indication to a user highlighting a region of the animal which requires further attention if present or provides a visual indication to the user highlighting changes between the historical reference image and a current image if present.

2. The apparatus of claim 1, wherein the camera is handheld and portable.

3. The apparatus of claim 1, wherein the camera weighs no more than 5 pounds.

4. The apparatus of claim 1, wherein the display content is cast to an additional screen.

5. The apparatus of claim 1, wherein the database of animal outlines comprises an outline of:
a. a canine;
b. a feline;
c. a bird;
d. a reptile;
e. an equine;
f. a swine;
g. a bovine;
h. a fish;
i. a marine mammal; or
j. an exotic pet.

6. The apparatus of claim 1, wherein the database of animal outlines comprises outlines of a front view, a rear view, a left side view, a right side view, a top view, and a bottom view for each animal.

7. The apparatus of claim 1, wherein the database of animal outlines comprises specific animal images, wherein the specific animal images comprise at least one of: a front view, a rear view, a left side view, a right side view, a top view, or a bottom view of the specific animal.

8. A method of using the apparatus of claim 1, comprising the steps of:
a. providing the apparatus of claim 1 in communication with at least one display;
b. allowing a user to select a category of animal;
c. providing an outline of the category of animal on the screen, wherein the outline is at least one of: a front view, a rear view, a left side view, a right side view, a top view, or a bottom view;
d. capturing an image of the animal; and
e. identifying an area of interest on the outline of the category of animal if an area of interest exists.

9. The method of claim 8, wherein capturing the image of the animal includes capturing an infrared image, a visible image, or both an infrared image and a visible image.

10. The method of claim 8, wherein identifying the area of interest comprises the steps of:
a. overlaying the captured image upon the outline of the animal;
b. comparing a captured thermographic signature with either: an expected thermographic signature or a historical thermographic signature; and
c. highlighting an area of interest wherein at least a portion of the captured thermographic signature does not match either the expected thermographic signature or the historical thermographic signature.
